(19) United States
(12) Patent Application Publication
(10) Pub. No.: US 2009/0105339 A1
(43) Pub. Date: Apr. 23, 2009

(54) Curative drug for neurodegenerative diseases

(76) Inventors: Satoru Sugiyama, Nagoya-shi (JP); Tokutaro Miki, Hachioji-shi (JP); Hiroshi Nishikawa, Shizuoka-shi (JP)

Correspondence Address: WENDEROTH, LIND & PONACK, L.L.P., 1030 15th Street, N.W.,, Suite 400 East, Washington, DC, 20005-1503, US

(21) Appl. No.: 12/230,667

(22) Filed: Sep. 3, 2008

(30) Foreign Application Priority Data
Oct. 23, 2007 (JP) 2007-275790

(51) Int. Cl. A61K 31/122; A61K 31/19

(52) U.S. Cl. 514/557; 514/688

(57) ABSTRACT

A compound is prepared based on Formula 1:

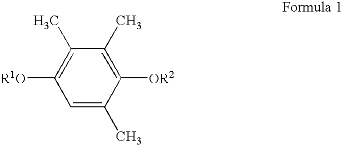

wherein, R¹ represents an alkyl group with a carbon number of 4 to 8, and R² represents a hydrogen atom, alkylcarbonyl group with a carbon number of 2 to 6 or alkoxycarbonyl group with a carbon number of 2 to 6. A curative drug for neurodegenerative diseases can be prepared principally from the compound or a hydroquinone derivative consisting of a cyclodextrin inclusion compound thereof. The curative drug for neurodegenerative diseases has a safe and beneficial effect of inhibiting nerve cell disorder due to oxidative stress, thereby to stop progression of symptoms.

### BACKGROUND OF THE INVENTION

1. Field of the Invention

This invention relates to a curative drug for neurodegenerative diseases, and particularly to preventive and curative drugs for neurodegenerative diseases such as Alzheimer's disease and Parkinson's disease.

2. Description of Related Art

There is a pressing need to develop preventive and curative drugs for neurodegenerative diseases such as Alzheimer's disease and Parkinson's disease, which increase in critical frequency with advancing age.

In the neurodegenerative diseases, different body sites on the body are affected, but diseases such as nerve cell degeneration and cell mortality are found anywhere on these sites. The studies on the cause and mechanism of the diseases have continued still now, but they remain to be explained. As curative drugs for those diseases, there have been used a cholinesterase inhibitor for Alzheimer's disease, and drugs for inhibiting dopamine degradation such as L-dopa preparation, dopamine-releasing promotor or COMT inhibitor for Parkinson's disease. However, these drugs cannot inhibit impairment of neuronal cells and are ineffective at stopping progression of symptoms.

Meanwhile, involvement of oxidative stress has been singled out as a possible cause of these diseases. The undermentioned Non-patent Literature 1 discloses the possibility of decreasing the oxidative stress, which results from various causes such as beta-amyloid having vitamin E accumulated in plenty in the brain of an Alzheimer's disease sufferer, ischemia reperfusion injury or aluminum accumulation, to cause a cerebroprotective action, thereby to slow the progression of Alzheimer's disease. The undermentioned Non-patent Literature 2 reports an effect of retarding progression to profound dementia, which can be brought about by administering to a moderate Alzheimer's disease sufferer high-dose vitamin E and selegiline which is an antioxidant agent used as an antiparkinsonism drug in a clinical experiment.

Non-patent Literature 1: Koichi Abe et al. “Vitamin” Vol. 74, No. 3, pp 113-119(2000)

Non-patent Literature 2: M. Sano et al., N Engl J Med, 336, 1216-1222(1997)

Although the administration of the antioxidant agent shows a likelihood and effect of slowing the progression of condition of the neurodegenerating disease to some extent, prevention of disease and significant improvements in disease presentation cannot be expected. Consequently, it is awkward to say that sufficient effect can be acquired. Thus, there has been a call for development of new drugs for the neurodegenerative disease, which has a beneficial effect capable of inhibiting nerve cell disorder due to the oxidative stress, thereby to stop progression of symptoms.

In the meanwhile, there is a compound formulated by the following general Formula 1, which specifically has a highly antioxidative effect and biocompatibility among hydroquinone derivatives having the antioxidative effect. In specific, this compound has more antioxidative activity than an antioxidant agent such as vitamin E and a potent effect of suppressing the production of nitric oxide (NO), which leads to increase in oxidative stress. An invention with respect to the compound and compositions using the compound, such as an oxidation inhibitor, therapeutic agent for refractory inflammatory diseases, cancerogenesis inhibitor, and therapeutic composition for arteriosclerosis is disclosed.

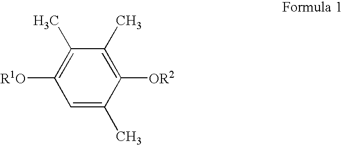

(wherein, R¹ represents an alkyl group with a carbon number of 4 to 8, and R² represents a hydrogen atom, alkylcarbonyl group with a carbon number of 2 to 6 or alkoxycarbonyl group with a carbon number of 2 to 6.)

As one example, Patent Literature 1 enumerated below discloses an antioxidant agent consisting of the aforesaid compound as an active constituent This citation further discloses that the antioxidant agent has more antioxidant properties than butylhydroxytoluene which is a typical food antioxidant agent and has lower toxicity to a biologic body.

Patent Literature 2 drafted by the inventors of this invention discloses the aforesaid compound has the antioxidative effect to block production of NO, thus to have efficacy as the therapeutic agent for refractory inflammatory diseases such as arthritis rheumatoides and nonspecific inflammatory bowel disease. Patent Literature 3 discloses a cancerogenesis inhibitor containing the aforesaid compound as an active constituent Patent Literature 4 drafted by the inventors of this invention discloses therapeutic composition for liver disorder containing the aforesaid compound as an active constituent. Further, Patent Literature 5 drafted by the inventors of this invention discloses the aforesaid compound has an effect of preventing cholesterol oxidation, which is exerted in vivo as a therapeutic agent for arteriosclerosis, and can be used safely.

It is reported in Non-patent Literature 3 that the aforesaid compound having a carbon number R¹ of 4 to 8 in an alkyl group typically has high antioxidative properties; in particular, the compound having the carbon number of 6 shows the highest antioxidative properties. Non-patent Literature 4 discloses that 2,3,5-trimethyl-hydroquinone-1-hexylether among the hydroquinone derivatives expressed in the general Formula 1 above has antioxidative properties twice as strong as vitamin E and a NO-production inhibitory activity 500 times as strong as vitamin E.

However, none of Patent Literatures 1 to 5 and Non-patent Literatures 1 to 4 disclose consideration of the behavior of the aforesaid compound on nerve cells and the inhibitory effect of the aforesaid compound on nerve cell disorder and practical exemplification of the aforesaid compound to be used as a curative drug for neurodegenerative diseases.

Patent Literature 6 discloses that 2-isopropylhydroquinone, which is a kind of hydroquinone derivatives, but not pertinent to the aforesaid compound as expressed in the general Formula 1, has a function of promoting biosynthesis of a nerve growth factor. However, this does not describe functions other than the biosynthesis promoting function, particularly the effect of suppressing oxidative stress. There is no description about which derivative among the hydroquinone derivatives is the most effective as a curative drug for nerve disease from the standpoint of a pharmacologic activity and biocompatibility.

Patent Literature 1: Japanese Unexamined Pat. Appln. Publication HEI 05-301836

Patent Literature 2: Japanese Unexamined Pat. Appln. Publication No. 2004-352661

Patent Literature 3: Japanese Unexamined Pat Appln. Publication HEI 06-100441

Patent Literature 4: Japanese Unexamined Pat Appln. Publication HEI 08-67627

Patent Literature 5: Japanese Unexamined Pat Appln. Publication No. 2002-241366

Patent Literature 6: Japanese Examined Pat. Appln. Publication HEI 7-110812

Non-patent Literature 3: Y. Nihro et al., Chem pharm Bull, 42, 576-579(1994)

Non-patent Literature 4: Wei Liu et al., J Pharm Pharmacol, 54, 383-389(2002)

### SUMMARY OF THE INVENTION

From the aforesaid standpoint, the inventors of this invention were made repeated keen studies on the effectiveness of hydroquinone derivative for neurodegenerative diseases, consequently to reveal that the hydroquinone derivative expressed in the aforesaid general Formula 1 has potent specific effect of protecting nerve cells from oxidative stress. Thus, the present invention was achieved on the basis of the revealed efficacy as a curative drug for neurodegenerative diseases.

Accordingly, it is an object of the present invention to provide a curative drug for neurodegenerative diseases, containing, as an active constituent, a compound represented by Formula 1 or a hydroquinone derivative consisting of a cyclodextrin inclusion compound thereof:

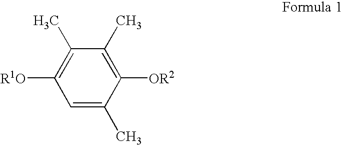

wherein, R¹ represents an alkyl group with a carbon number of 4 to 8, and R² represents a hydrogen atom, alkylcarbonyl group with a carbon number of 2 to 6 or alkoxycarbonyl group with a carbon number of 2 to 6.

Another object of the invention is to provide the curative drug for neurodegenerative diseases as described above, in which the compound represented by the aforementioned Formula 1 is 2,3,5-trimethyl-hydroquinone-1-hexylether or 2,3,5-trimethyl-hydroquinone-1-hexylether 4-acetate.

The hydroquinone derivative according to the invention, which consists of the compound represented by the aforementioned Formula 1 or cyclodextrin inclusion compound has an effect of strongly protecting nerve cells from oxidative stress and can be used safely. Thus, the composition containing the hydroquinone derivative as an active constituent according to the invention can be effectively used as the curative drug for neurodegenerative diseases.

In particular, the compound consisting of 2,3,5-trimethyl-hydroquinone-1-hexylether or 2,3,5-trimethyl-hydroquinone-1-hexylether 4-acetate according to the invention is excellent in terms of a pharmacologic activity and biocompatibility, and therefore, can be used more effectively.

Further advantages and specific details of the invention will be set forth hereinafter in conjunction with the following detailed description of a presently preferred embodiment.

### DESCRIPTION OF THE PREFERRED EMBODIMENTS OF THE INVENTION

The invention will be better understood and objects other than those set forth above will become apparent when consideration is given to the following detailed description thereof.

In the compound contained in the curative drug for neurodegenerative diseases according to the invention, which is represented by the aforementioned Formula 1, the alkyl group with a carbon number of 4 to 8 indicated by R¹ may optionally assume a straight chain, branched chain or cyclic structure. For instance, there may be various types of butyl group, pentyl group, hexyl group, heptyl group, octyl group, cyclobutyl group, cyclopentyl group, cyclohexyl group, cycloheptyl group, and cyclooctyl group. From the aspect of a pharmacologic activity and biocompatibility, the compound having a straight-chain structure with a carbon number of 4 to 7 is preferable, and the hexyl group is particularly preferable.

The alkylcarbonyl group with a carbon number of 2 to 6 in R² may assume a straight chain structure or a branched chain structure. For instance, acetyl group, propionyl group, butyryl group, and isobutyryl group can be enumerated. Also, the alkoxycarbonyl group with a carbon number of 2 to 6 in R² may be a straight chain structure or a branched chain structure. For instance, methoxycarbonyl group, ethoxycarbonyl group, propoxycarbonyl group and isopropoxycarbonyl group can be enumerated.

As a preferable compound as represented by Formula 1 above in terms of a pharmacologic activity, 2,3,5-trimethyl-hydroquinone-1-butyl ether, 2,3,5-trimethyl-hydroquinone-1-hexylether, and 2,3,5-trimethyl-hydroquinone-1-hexylether 4-acetate can be enumerated.

As one example, the compound as represented by Formula 1 and the cyclodextrin inclusion compound thereof can be produced by a method described in the aforesaid Patent Literature 5.

The curative drug for neurodegenerative diseases according to the invention contains, as an active constituent, as an active constituent, a compound represented by Formula 1 or a hydroquinone derivative consisting of a cyclodextrin inclusion compound thereof, and is prepared by adding pharmaceutically approved additives such as pharmaceutical carrier and excipients. The curative drug of the invention may assume many and varied forms as conventionally applied for medicinal purposes, for example, in a orally-bioavailable form capable of being readily absorbed from the digestive tract such as of a tablet, granulated powder, capsules or liquid medication, a parenteral form such as a transdermal absorption drag as prepared in a injectable liquid or suppository form, or a soluble solid or liquid form which can be dissolved with a suitable solvent in use in consideration for circulation and storage stability. Furthermore, application of a technique for a blood-brain transferring mechanism is especially useful for the curative drug for neurodegenerative diseases of the invention.

A dose of the curative drug for neurodegenerative diseases of the invention depends on various factors such as an intended curative effect, administration method, age and weight of a subject Therefore, the dose of the curative drug is not definitively determined, but a daily dosage for parenteral administration is generally about 0.01 to 100 mg, preferably 0.05 to 10 mg, per kilogram of body weight. For oral administration, a daily dosage is about 0.1 to 300 mg, preferably 0.5 to 100 mg. The prescribed amount of curative drug may be orally administered daily at a time or in two to five divided doses.

### Embodiments

The medical experiment carried out according to the present invention will be described hereinafter in detail, but the invention should not be understood as being limited to the experiments.

[Experimental Example: Cytoprotective Properties with Cultured Cells]

Of the hydroquinone derivatives expressed in the aforesaid Formula 1, 2,3,5-trimethyl-hydroquinone-1-hexylether (Compound 1) was examined in comparison with vitamin E with respect to the activity of inhibiting toxicity of lipid peroxide to nerve cells. PC12 cells as a cell culture model of for human neuroblastoma-derived SH-SY5Y and rat pheochromocytoma cells were cultivated by the law of the art. A culture media was prepared in effect by adding FCS (fetal calf serum) to Dulbecco's Eagle medium (DMEM) and used further with the addition of NaHCO₃ (3.7 g/L), penicillin G (100 U/mL) and Streptomycin (100 mg/ml). A cultivation was performed by seeding 6×10⁴ SH-SY5Y cells and 4×10⁴ PC12 cells in a CO2 incubator at 37° C. using a microplate with 24 macroscopic pores. After 48 hours, the aforesaid Compound 1 or vitamin E was added thereto. Then after about 30 minutes, 70 μM of linoleic acid hydroperoxide were added thereto for the SH-SY5Y cells and 35 μM of the same were added for PC12 cells. After cultivation for about 20 hours, the number of surviving cells was measured.

The measurement of the surviving cells was made by a Methylene blue dye uptake method. To be more specific, the cells were rinsed twice with PBS (phosphate buffered saline free from Mg and Ca), and then, fixated with 10% formalin for 5 minutes. Further, the cells were rinsed three times with distilled water, stained with 0.05% methylene blue solution for 60 minutes, and rinsed three times with distilled water. Then, 20 minutes after adding 0.33N HCl thereto, the absorbance of the cells was measured at 665 nm. The survival rate and protection rate of the cells were calculated on the basis of the measured result by the following equation.

Cell survival rate (%)=[absorbance of treated cells with testing additives]/[absorbance of non-treated cells]×100

Protection rate (%)={1−(100−[survival rate of treated cells with testing additives])/(100−[survival rate of cells with no additives])}×100

The experimental results on SH-SY5Y cells are shown in Table 1 below, and the experimental results on PC12 cells are shown in Table 2 below.

TABLE 1
Effect on cell disorder due to linoleic acid hydroperoxide
(LA-OOH) on SH-SY5Y cells

| | Dosage | LA-OOH | Survival | Protection |
|---|---|---|---|---|
| Test Substance | (μM) | (μM) | Rate (%) | Rate (%) |
| Vitamin E | 0 | 0 | 100.00 | — |
| | 0 | 70 | 80.97 | 0.0 |
| | 0.5 | 70 | 83.72 | 14.5 |
| | 1.0 | 70 | 79.77 | −6.3 |
| | 5.0 | 70 | 79.78 | −6.2 |
| | 10.0 | 70 | 81.78 | 4.3 |
| Compound 1 | 0 | 0 | 100.00 | — |
| | 0 | 70 | 80.52 | 0.0 |
| | 0.5 | 70 | 83.41 | 14.8 |
| | 1.0 | 70 | 84.83 | 22.1 |
| | 5.0 | 70 | 87.97 | 38.3 |
| | 10.0 | 70 | 87.04 | 33.5 |

Compound 1: 2,3,5-trimethyl-hydroquinone-1-hexylether

TABLE 2
Effect on cell disorder due to linoleic acid hydroperoxide
(LA-OOH) on PC12 cells

| | Dosage | LA-OOH | Survival | Protection |
|---|---|---|---|---|
| Test Substance | (μM) | (μM) | Rate (%) | Rate (%) |
| Vitamin E | 0 | 0 | 100.00 | — |
| | 0 | 35 | 32.61 | 0.0 |
| | 0.1 | 35 | 37.16 | 6.8 |
| | 0.5 | 35 | 35.88 | 4.9 |
| | 1.0 | 35 | 35.85 | 4.8 |
| | 5.0 | 35 | 42.57 | 14.8 |
| Compound 1 | 0 | 0 | 100.00 | — |
| | 0 | 35 | 30.76 | 0.0 |
| | 0.1 | 35 | 45.68 | 21.5 |
| | 0.5 | 35 | 86.57 | 80.6 |
| | 1.0 | 35 | 91.96 | 88.4 |
| | 5.0 | 35 | 92.31 | 88.9 |

Compound 1: 2,3,5-trimethyl-hydroquinone-1-hexylether

It is apparent from Table 1 and Table 2 that Compound 1 exerts an effect of validly restraining cytopathy due to linoleic acid hydroperoxide on both the SH-SY5Y cells and PC12 cells in comparison with vitamin E and advantageously has lower toxicity.

The protection rate of Compound 1 added to the cells is far higher than a value expected merely from a cell protecting effect from oxidative stress due to an antioxidative effect, i.e. an expected value predictable from the fact that the antioxidative effect of Compound 1 is double that of vitamin E. Particularly, the survival rate and protection rate of PC12 cells according to Compound 1 both are up to around 90%, which is notably higher than vitamin E showing a survival rate up to about 42% and a protection rate of around 15% for the PC12 cells. The cause is still not known, but it is assumed that the hydroquinone derivative has possibly behaviors contributing to protection and survival of nerve cells, other than the antioxidative effect That is, as one of those effective behaviors, it has been reported that the hydroquinone derivative is considered to have a promotive effect for stimulating biosynthesis of nerve growth factor. The survival rate and protection rate of the cells may possibly be increased due to a synergy of the antioxidative effect and the cellular protection effect, promoting growth and survival of the nerve cells. The exceptional effects of the Compound 1 of the invention are clearly beneficial as the curative drug for neurodegenerative diseases.

From the experimental results as described above, it is evident that the compound according to the present invention is significantly effective for protecting neuronal cells from oxidative stress due to lipid peroxide.

While the invention has been explained by reference to particular embodiments thereof, and while these embodiments have been described in considerable detail, the invention is not limited to the representative medicinal substance and medicine manufacture as described Those of ordinary skill in the art will recognize various modifications which may be made to the embodiments described herein without departing from the scope of the invention. Accordingly, the scope of the invention is to be determined by the following claims.

1. A curative drug for neurodegenerative diseases, containing, as an active constituent, a compound represented by Formula 1 or a hydroquinone derivative consisting of a cyclodextrin inclusion compound thereof: 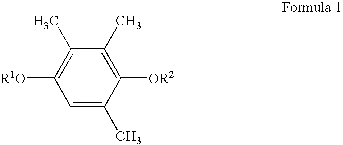
wherein, R¹ represents an alkyl group with a carbon number of 4 to 8, and R² represents a hydrogen atom, alkylcarbonyl group with a carbon number of 2 to 6 or alkoxycarbonyl group with a carbon number of 2 to 6.

2. The curative drug for neurodegenerative diseases as set forth in claim 1, wherein said compound represented by said Formula 1 is 2,3,5-trimethyl-hydroquinone-1-hexylether or 2,3,5-trimethyl-hydroquinone-1-hexylether 4-acetate.

3. A method for treating a neurodegenerative disease which comprises administering, to a patient in need thereof, a therapeutically effective amount of a compound represented by Formula 1 or a hydroquinone derivative consisting of a cyclodextrin inclusion compound thereof: 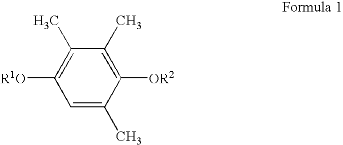
wherein, R¹ represents an alkyl group with a carbon number of 4 to 8, and R² represents a hydrogen atom, alkylcarbonyl group with a carbon number of 2 to 6 or alkoxycarbonyl group with a carbon number of 2 to 6.

4. The method as set forth in claim 3, wherein said compound represented by said Formula 1 is 2,3,5-trimethyl-hydroquinone-1-hexylether or 2,3,5-trimethyl-hydroquinone-1-hexylether 4-acetate.
